(19) United States
(12) Patent Application Publication
(10) Pub. No.: US 2007/0010752 A1
(43) Pub. Date: Jan. 11, 2007

(54) System and method for analysing the p-wave of an ecg-signal

(76) Inventor: Pentti Korhonen, Kangasala (FI)

Correspondence Address: VENABLE LLP, P.O. BOX 34385, WASHINGTON, DC, 20043-9998, US

(21) Appl. No.: 10/552,697

(22) PCT Filed: Apr. 2, 2004
(86) PCT No.: PCT/FI04/50034

§ 371 Date: Jul. 27, 2006

(30) Foreign Application Priority Data
Apr. 10, 2003 (FI) 20030547

(51) Int. Cl. A61B 5/04

(52) U.S. Cl. 600/516

(57) ABSTRACT

The invention relates to a method, to a system and to a computer program product for the cardiac analysis. The method comprises steps for acquiring the ECG-signal in the form of vectorcardiography, detecting wave from the EGG-signal by a template method and calculating the parameter values of the P-wave preferably continuously during the ECG-recording. The method is aimed to the dynamic changes of the R wave, wherein substantially every detected P wave is compared to the reference P-wave in defined time period. The cardiac analysis system according to the invention is configured to implement the aforementioned method. The computer program product comprises computer program code for implementing the aforementioned method.

The invention relates to a method according to the preamble of the appended independent claim 1 for cardiac analysis, which method comprises steps for acquiring an ECG-signal, detecting at least one wave of the ECG-signal and calculating parameter values of said wave. The invention also relates to a cardiac analysis system according to the preamble of the appended independent claim 19 for implementing the aforementioned method as well as to a computer program product according to the preamble of the appended independent claim 32.

### BACKGROUND OF THE INVENTION

An electrocardiogram (ECG) is a record of electrical events occurring within the heart of a patient and it is used for examination of heart diseases. The ECG plots the sequence of changes in electrical potential differences recorded between different regions of the patient's body surface. The ECG can be measured using various lead systems known as such. Commonly the ECG is obtained by using a standard 12-lead arrangement, but it can be obtained by using other lead systems, for example a Frank system, which uses three leads. These ECG-measurements provide a one-dimensional electrocardiographic signal, but it is also possible to present ECG as a three-dimensional signal, a so-called vectorcardiography.

The basic form of the ECG-signal can be seen in FIG. 1. The ECG breaks down each heartbeat into a series of electrical waves. Three of the waves (P, QRS, T in the figure), a P-wave, a QRS-complex and a T-wave, are associated with the heart's contractions. The first deflection of the ECG is called the P-wave. It reflects the sequential activation (depolarization) of the heart's upper chambers, the right and the left atria. After the P-wave the ECG returns to its baseline, which is a substantially straight line on an ECG-paper where there are no positive or negative changes of electricity to create deflections. The next is a complex called QRS-complex, which reflects the sequential activation of the heart's lower chambers, the right and the left ventricle.

After the ORS-complex, the ECG returns to, or very nearly to, its baseline which is called a ST-segment, where it remains until the appearance of a T-wave. The T-wave reflects ventricular repolarization.

When discussing the ECG, a term “interval” refers to the length of the wave along with an isoelectric line following it. The interval can be named by using the letters of both waves on either side (e.g. PQ). A space between dash lines 1 and 3 (FIG. 1) represents the PQ-interval. A term “segment” refers to the baseline between the end of one wave and the beginning of the next wave. The space between dash lines 2 and 3 represents the PQ-segment, as well as the space between dash lines 4 and 5 represent the ST-segment.

The above-mentioned vectorcardiogram (VCG) is a form of the electrocardiography that represents movements of vectors of the myocardial activation with direction and magnitude as loops in a three-dimensional space. Vectorcardiography can be considered as a method of recording the direction and magnitude of electrical forces of the heart by means of a continuous series of vectors that form a curving line around a center. A spatial orientation and magnitude of the heart vector is projected onto three orthogonal planes as frontal, horizontal and sagittal planes. The tip of the vector with coordinates (X, Y, Z) should trace out a loop in space, called a vector loop. The vector loop starts from the zero-point, which corresponds to the isoelectric line or the baseline on a usual scalar ECG. The loop consists of, similar to the ECG-signal, a P-loop, a QRS-loop and a T-loop. When heart cycle is completed, the QRS-loop returns, in a normal situation, to zero-point and closes the loop. On the other hand, if the patient has a ST-change, the QRS-complex will not end to the baseline, but over or under, depending on whether the patient has a ST-elevation or depression.

The general features of the QRS-complex and the ST-segment within the ECG-signal have been extensively studied and their diagnostic significance is quite well known. On the contrary, far less attention has been paid to the P-wave and therefore the clinical properties of this waveform are still not fully understood. It is known that the P-wave represents atrial depolarization and it can show the heart's rate and rhythm. It is known that the atrial abnormalties and the P-waves are related to some heart diseases, for example to heart failure or to the susceptibility to atrial arrhythmias. Still, the specificity of these changes has been low.

Developed ECG-systems observe the QRS-complex and the ST-segment ideally, but have a lack in their operation of observing the P-wave. A reason for that can be found, in common opinion, where the P-wave and the atrias have been considered less meaningful compared to the QRS-wave, the ST-segment and the ventricles. Due to this, there are significantly less studies concerning the P-wave. In addition, the known studies of the P-wave use mostly the known 12-lead ECG. Such a 12-lead ECG-arrangement has a disadvantage in expression of the waves; it does not give a good three-dimensional picture of the electrical waves, which neglects the small waves, for example the P-wave. Studies done of the P-wave utilize mostly the ECG recording system that expresses the heart situation in one moment, but is not capable of continuously showing the in-time coming dynamic changes of the examined P-wave.

### SHORT DESCRIPTION OF THE INVENTION

The present invention relates to a cardiac analysis method and system, which takes into account said lacks in existing systems. More precisely, the cardiac analysis method according to the invention comprises the steps for acquiring the ECG-signal, detecting at least one wave of the ECG-signal and calculating the parameter values of said wave, which is the P-wave. The cardiac analysis method according to the invention is then focused to the dynamic changes of the P-wave. The cardiac analysis system according to the invention comprises means for acquiring the ECG-signal, means for detecting at least one wave of the ECG-signal and means for calculating the parameter values of said wave, which is the P-wave. The system is additionally adapted to focus the analysis to the dynamic changes of the P-wave, wherein said system also comprises means for comparing substantially every detected F-wave to the reference P-wave in defined time period. The computer program product according to the invention comprises computer readable instructions for detecting at least one wave from the ECG-signal, which said wave is a P-wave, whereupon the computer program code comprises computer readable instructions for focusing to the dynamic changes of the P-wave, wherein said computer program code additionally comprises computer instructions configured to compare substantially every detected P-wave to a reference P-wave in defined time period.

In accordance with the invention, the ECG-signal is processed in the form of vectorcardiogram that can be described by three orthogonal leads: X, Y, Z. P-wave is at first recognized by a template and then the signals of the P-wave are averaged to form a smooth wave form, which is used to determine the P-wave specific parameter values continuously from the whole ECG-recording. The cardiac analysis method according to the invention is then addressed to the dynamic changes of said P-wave. That means that it addresses to the changes of P-wave configuration that happen smoothly in the follow-up time. The method comprises means for comparing substantially every detected P-wave to the reference P-wave in a defined time period. The results from each parameter are presented as points on a trend graph. The cardiac analysis method according the invention also provides means to notice and pick up the atrial extrasystoles, to separate them into different sub-classes based on their morphology and then to average and analyze them in the same way as the P-wave.

The analysis of the P-wave according to the invention, brings new tools to the analysis of atrial activation; In consequence the P-wave is considered to be more meaningful than before. With the method according to the invention, there is a first-time chance to see the dynamic changes of P-wave in a patient who has an acute myocardial infarction that affects also the atrial tissue. The atrial infarction is the entity that is supposed to affect the patient outcome by causing atrial arrhytmias and ruptures of the atrial free wall in special situations. Still, the dynamic changes in the P-wave configuration and the diagnostic criteries of the atrial infarction are unclear.

The analysis of the P-wave according to the invention provides also an effective way to monitor atrial arrhythmias, especially the paroxysmal atrial fibrillation. The atrial fibrillation is the most common cardiac arrhythmia, the prevalence of which is increasing with the aging of the population. Because of its clinical importance and the lack of highly satisfactory management approaches, it is the subject of active clinical and research efforts. With the method according to the invention, it is possible to observe the dynamic changes of P-wave after the cardioversion when the normal sinus rhythm has been achieved and to try to find explanations for why some of the patients will maintain sinus rhythm after the cardioversion and some of them will not. Additionally, with the method according to the invention, it is possible to observe the dynamic changes of P-wave in situations where the risk of atrial fibrillation is high, for example after heart surgery. Because the method of the invention is based on dynamic changes of the P-wave, it also gives a possibility of observing the actions of treatment and medicine that is given to maintain sinus rhythm.

The analysis of the P-wave according to the invention also provides an effective way to monitor the dynamic changes of P-wave in the patient who has an acute heart insufficiency. These P-wave abnormalities are discussed in more detail later in the description. It is obvious that with the method according to the invention, it is possible to observe the dynamic changes of P-wave also in other situations where the atrias are in the changing abnormal situation that affects the P-wave configuration as well.

The preferred embodiment of the invention is set forth in the drawings, in the detailed description which follows, and in the annexed claims. Further objects and advantages of the invention are also considered in the description. The invention itself is defined with particularity in the claims.

### BRIEF DESCRIPTION OF THE DRAWINGS

FIG. 1 is a graphical representation illustrating the ECG-signal,

FIG. 2 illustrates a flow chart of the steps of the method according to the invention,

FIG. 3 illustrates a flow chart of the steps of the P-wave analysis according to the invention,

FIG. 4a-c illustrates the P-wave in one-dimensional environment,

FIG. 5a-b illustrates the P-wave vector loop in two-dimensional environment,

FIG. 6a-b illustrates the P-wave vector loop in three-dimensional environment,

FIG. 7 illustrates the P-wave in magnitude ECG, and

FIG. 8a-b illustrates the change area, the change vector angle and magnitude.

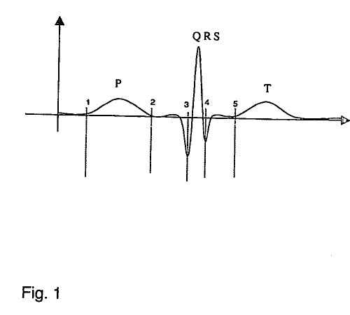

### DETAILED DESCRIPTION OF THE PREFERRED EMBODIMENT

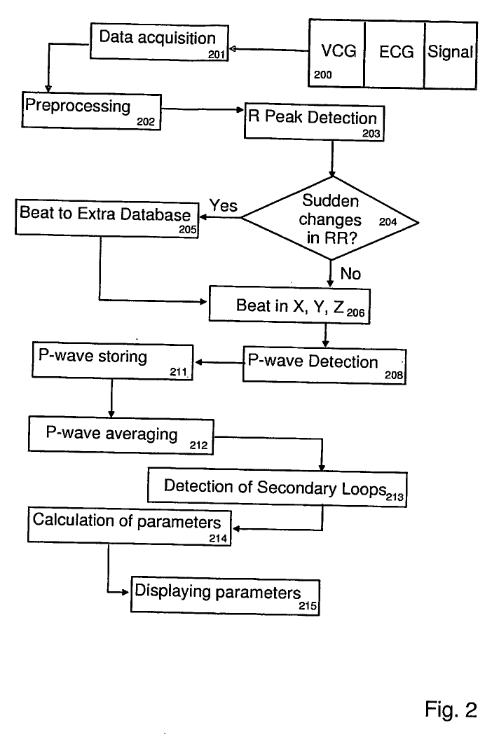

FIG. 2 illustrates the steps of the method according to the invention as a flow chart. The present invention exploits the known three-dimensional electrical ECG-model called vectorcardiogram. ECG signal acquisition (201) can be done in various ways. A basic method is to use eight standard ECG-surface electrodes, which are placed on the patient according to the Frank electrode system. The electrodes are then used to form an ECG-vectorcardiogram by method known in prior art, which ECG-vectorcardiogram can be described by three orthogonal leads X, Y, Z. It is also possible to acquire the ECG-signal by using a standard twelve-lead-ECG-arrangement, which is stored and derived to form a vectorcardiogram and then further analyzed. According to the invention, the ECG-signal is acquired preferably from existing vectorcardiogram data collected by another system (200). For example, the ECG-signal can preferably be acquired from the MIDA data storage unit. The MIDA is a commercial monitoring system (sold by Philips Medical) made to analyze the ischemic changes of QRS-complex and ST-segment of ECG and it is widely used in hospitals. MIDA registers the electrical signals of the heart using said Frank electrode system and constructs the three-dimensional electrical model from them. The action of MIDA is discussed in more detail in U.S. Pat. No. 5,520,191. It is obvious that the ECG-signal in the present invention can be acquired by not only the mentioned but any known methods.

After the acquisition, the ECG-signal is preprocessed (202) to minimize electrical artefacts. Preferably the raw data is filtered to remove the noise and to improve its signal-to-noise-ratio (SNR). The noise is filtered by some known signal processing method, which are not discussed more in this as it can be considered obvious to the man skilled in signal processing.

The preprocessed signal is according to the invention analyzed to detect the peaks of R-waves (203). A gap between two peaks is referred to by a term “beat”. When the peaks are found, a time period between them is measured and the beat is stored. Beats are classified depending on their duration (time period). If the time between two consecutive R-waves changes suddenly under predetermined time, it can be assumed that the beat consists of an atrial extra systole (204). In that case the beat is stored to the atrial extra systole database for further analysis (205). The atrial extra systoles can be categorized into subclasses according to their morphology. Afterwards, they are averaged and analyzed. The result of this examination will give information of the changes of the atrial extra systoles as well as the number of the atrial extra systole sub-groups. By picking the extra systoles from the ECG-signal, the resulting P-wave data will become as homogenous as possible, which is preferable to the analysis. There is also a possibility that the change results from the ventricular extra systole when there is no atrial extra systole preceding the deviant-looking QRS-complex. In this case, the changed beat is also picked to the second database and later removed. All the other beats (the unchanged, time period within the predetermined time limit) are stored to a P-wave database. The beats in said both databases are stored in the X, Y, Z -leads (206) and used separately for the P-wave analysis according to the invention.

P-wave Analysis:

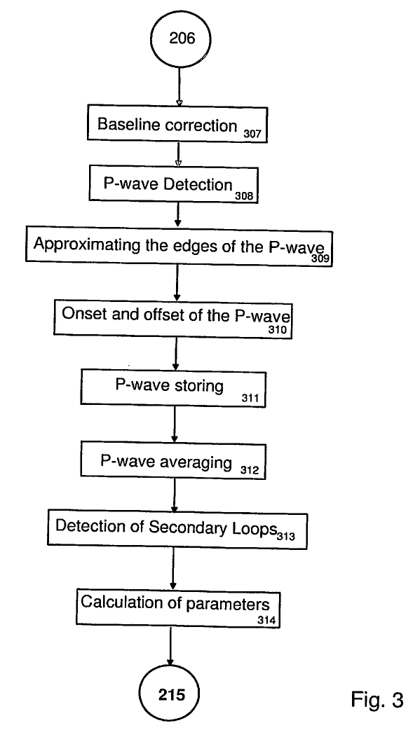

Reference is now made to FIG. 3, in which the examination of the P-wave is described in more detail. At first the P-wave signal is processed by baseline correction (307) to minimize the baseline drift of the signal. This way the signal quality will improve. There are several methods for implementing the baseline correction, for example linear interpolation, quadratic or cubic corrections, polynomial fitting and high pass frequency filtering. Because said methods are known as such, they are not explained further.

The P-wave is normally viewed as a smooth, small and curved deflection. It is usually a positive deflection, although it may be negative as well. The duration of the P-wave is normally below 0.12 seconds and the amplitude is normally less than 0.25 mV. In the frontal plane the P-wave axis angle ranges from 0 to 75 degrees. Also notched P-waves may be seen and the normal P-wave may often exhibit two components as an M-shaped complex. Because of its small, ill-defined and variable shape, the detection of the P-wave is difficult.

According to the invention, the P-wave detection (308) is preferably done by a template method, which is a type of two-dimensional cross-correlation method with both time and amplitude as variables. The template method attempts to detect the P-wave by using the covariance between the template signal and the wave form signal stored beat. For substantially each beat, the template is tried to be matched with the actual signal and the P-wave is detected when the covariance exceeds a specified threshold. The P-wave is valid if its duration is between the minimum and the maximum P-wave durations. As said, the length of the P-wave is generally about 120 ms. Time between the beginning of the P-wave to the beginning of the Q-wave (called PQ-time) is generally below 200 ms. These values are preferably used to form a template time window, but the time window can be predefined with some other values or with entering the values occasionally.

The P-wave detection is preferably carried out only in one coordinate, because frequently one coordinate is less noisy than the others and therefore more suitable for analysis. In the detection, the user is at first asked to enter the starting ad ending sample of the template in order to construct the first template. Later, the template can be changed if there is a need for it. It can be done manually in the same way as the first template. It can be done also automatically in that way that the predetermined amount of latest detected P-waves is taken and averaged, wherein the averaged P-wave is set to be the next template. The template is also checked for the adaptation, if the amount of dropped P-waves exceed the predetermined level. In these ways, the template can be changed according to the changes of the P-wave. In other words, template changes as the P-wave changes. Obviously, the P-wave can also be detected by some other methods known in the prior art, such as thresholding, patter recognition etc.

It has been empirically observed that the preprocessed ECG-signal can still be relatively noisy around the P-wave, which can affect the determination of the real location of the onset and offset of P-wave. The amplitude of the deflection might be affected by the existence of a noise plateaux before the beginning of the P-wave. For avoiding this an improved method of detection of the P-wave time limit is preferably used (309). In the improved method, the rising and falling edges of the P-wave are approximated by straight lines using a slope function. Later values of the baseline of the beat are calculated and stored. Intersection points of the baseline and the two edges are obtained by an intersection function.

With the help of the baseline, the value of the onset and the offset will be determined (310). The onset is the intersection of the baseline and the rising edge, whereas the value of the offset will be the intersection of the baseline and the falling edge. The value for the P-wave offset can also be found by a threshold method, in which the sample having the ECG of approximately the same value as in the P-wave onset is searched. The found value is accepted as the real offset of the signal based on the idea that in most of the cases the P-wave bop will be closed in three-dimensional environment, which means (in time domain) that the P-wave must return to its initial value.

The method of finding the P-wave onset and offset is extremely important in analyzing the P-wave specific parameters, because in those calculations the P-wave must have clear boundaries, in other words, a P-wave vector loop must be completed. The P-wave vector loop is discussed in more detail later in the description. However, it is equally important to notice that the offset function is not used while analyzing the PQ-segment specific parameters, because with those parameters the PQ-segment elevation, in other words the incomplete P-wave vector loop, must be analyzed in the unedited situation. Therefore, the P-wave onset function is used in the method substantially all the time, and the P-wave offset function is used with the P-wave specific parameters.

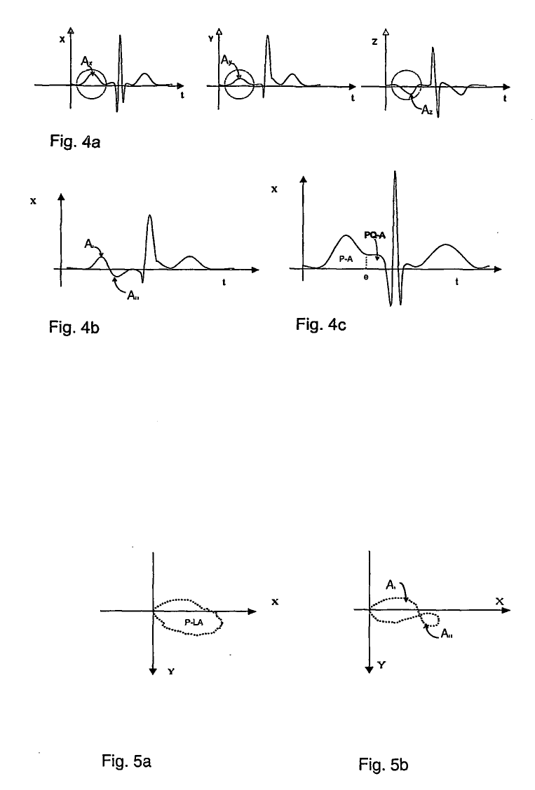

The detected P-wave is stored in X-, Y- and Z-leads (FIG. 4a, P-wave circled; FIG. 3, 311). All the P-wave beats are then averaged (312) in the predetermined time intervals to form a smooth beat. Formed beats are used for calculations and analysis. The first averaged P-wave is used as an initial reference P-wave, where the upcoming averaged P-wave is compared to (monitoring of the dynamic changes).

When monitoring the myocardial infarct, the patient is in cardiac monitor, e.g. MIDA, preferably for 1-2 days. Substantially all the ECG-signals during that time should be taken for the analysis. Averaging for the data is preferably done every 4 minutes.

When monitoring the cardioversion of the patient with atrial fibrillation, the normal sinus rhythm is observed preferably for 3-4 hours.

Averaging for the data acquired is preferably done every 10 sec-2 minutes. The reason for the shorter averaging time is due to the lesser data and the quicker changes in the beginning.

It is also possible to monitor the P-wave changes, for example, 1, 3 or 6 months after the procedure such as, for example, the cardioversion. In that case the monitoring may last 15-30 minutes and it focuses on observing the change of the vector loop. The different monitoring results are combined for better analysis.

It is obvious that the monitoring depends on the situation, when also the above-mentioned time limits vary according to the situation.

In some situations, the P-wave can be seen as two loops, the primary (Aₗ) and the secondary (Aₗₗ) loop, which are detected (313). The loops are illustrated in FIGS. 4b and 5b in one-dimensional and two-dimensional spaces. Detection of the secondary loop is essential in P-wave loop analysis, as it can be greatly valuable in the analysis of atrial anatomic changes described later in the text. P-wave loops in a three-dimensional space can preferably be projected onto the three orthogonal planes and the existence of the secondary loop can thus be completed in two dimensions.

The QRS-complex is also observed in the method according to the invention. This is done by detecting the beginning and the ending of the QRS-complex from the beginning of the beat. The duration of the QRS-complex is also measured, as well as the waveform signal. These values are used along the P-wave analysis.

According to the invention, the averaged P-waves are calculated and the parameters described later in the text are estimated next. (314). A few of the parameters are common parameters, several are developed for the invention and for the dynamic analysis of the P-wave, since they have not existed before. Parameters can be divided into four categories: one-dimensional environment, two-dimensional environment, three-dimensional environment and magnitude ECG environment.

One-Dimensional Environment

The parameters of a one-dimensional diagram describe the P-wave properties in one dimension and relate to the orthogonal axes of the electrocardiogram. FIGS. 4a and 4b illustrate the P-wave in a one-dimensional environment. A vector area (P-A) (FIG. 4a) expresses the area of the P-wave. It can be derived from the areas in the three orthogonal lads X, Y, Z to form an equation (sqrt corresponds to the square root):
P-A=sqrt(A_{X}²+A_{Y}²+A_{Z}²).

A vector change area (PC-A) describes the changes of the P-wave area. Preferably the area of the P-wave being examined (Aₑₓₐₘ) is compared with the area of the reference P-wave (A_{ref}). The difference is calculated from X, Y, Z-leads:
PC-A=sqrt[(Aₑₓₐₘ−A_{ref})_{X}²+(Aₑₓₐₘ−A_{ref})_{Y}²+(Aₑₓₐₘ−A_{ref})_{Z}²].

A P-area duplicity (P-AD) can be calculated by relating the area of the secondary loop of the P-wave (Aₗₗ) to the primary loop area (Aₗ) from X, Y, Z-leads (FIG. 4a).
P-AD=sqrt[(Aₗₗ/Aₗ)_{X}²+(Aₗₗ/Aₗ)_{Y}²+(Aₗₗ/Aₗ)_{Z}²].

In addition, the PQ-parameters are calculated. A PQ-vector magnitude (PQ-VM) describes the PQ-magnitude (level of PQ segment elevation) and a PQ-area (PQ-A) describes the area between the PQ-segment and the baseline (e) in a one-dimensional environment (FIG. 4c). A PQC-A-parameter describes correspondingly the upcoming changes of the PQ-area compared to the values of the reference P-wave. A PQ time and a P-wave duration (P-dur) are also calculated in the one-dimensional environment.
PQ-VM=sqrt(PQ_{X}²+PQ_{Y}²+PQ_{Z}²)
PQ-A=sqrt(PQA_{X}²+PQA_{Y}²+PQA_{Z}²)
PQC-A=sqrt[(Aₑₓₐₘ−A_{ref})_{X}²+(Aₑₓₐₘ−A_{ref})_{Y}²+(Aₑₓₐₘ−A_{ref})_{Z}²]
Two-Dimensional Environment:

The parameters of a two-dimensional diagram describe a P-wave vector loop in the three orthogonal planes: frontal (XY), horizontal (XZ) and sagittal (YZ) planes. The P-wave vector loop in a two-dimensional environment is shown in FIGS. 5a and 5b. A P-wave vector loop area (P-LA) is the area of a two-dimensional loop described by the P-wave vector (FIG. 5a). It can be derived from the different areas in the three orthogonal planes XY, XZ and YZ to form an equation:
P-LA=sqrt(A_{XY}²+A_{XZ}²+A_{YZ}²)

A P-wave change loop area (PC-LA) is the difference of the two-dimensional areas of the F-wave vector loop being examined and the reference P-wave vector loop:
P-LA=sqrt[(Aₑₓₐₘ−A_{ref})_{XY}²+(Aₑₓₐₘ−A_{ref})_{XZ}²+(Aₑₓₐₘ−A_{ref})_{YZ}²]

A vector loop area duplicity (P-LAD) describes the P-wave duplicity ratio, which is the ratio of the secondary loop (Aₗₗ) to the primary loop (Aₗ) (FIG. 5b):
P-LAD=sqrt[(Aₗₗ/Aₗ)_{XY}²+(Aₗₗ/Aₗ)_{XZ}²+(Aₗₗ/Aₗ)_{YZ}²]
Three-Dimensional Environment.

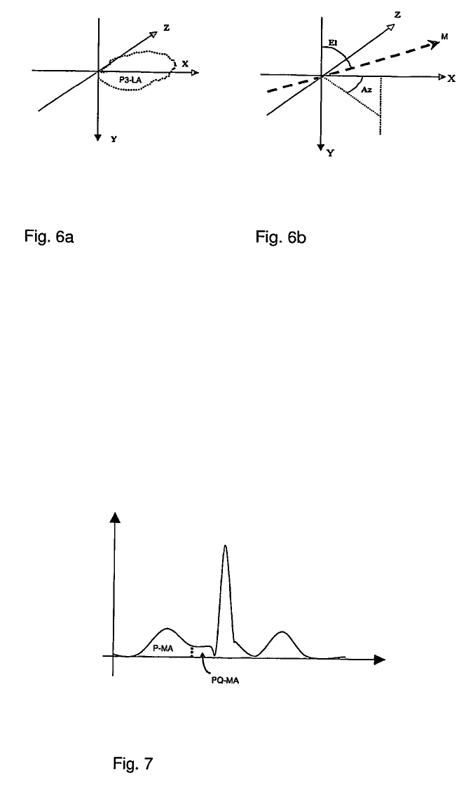

The parameters of a three-dimensional diagram describe the P-wave vector loop in the three-dimensional environment (FIG. 6a). A three-dimensional P-wave vector loop area (P3-LA) is the area of the three-dimensional loop described by the P-wave vector. The basic function, called “looparea”, calculates the area of a loop dividing it into small triangles in three dimensions and using the known vector cross product method to compute their areas. The three-dimensional P-change loop area (P3 C-LA) is the difference between the area of the P-wave vector loop being examined (Aₑₓₐₘ) and the reference P-wave vector loop (A_{ref}):
P3 C-LA=sqrt[(Aₑₓₐₘ−A_{ref})²].

A P-azimut (P-Az) is a parameter for the angle that a P-main vector (M) describes in the transversal plane (FIG. 6b). The P-main vector is defined as the average vector of substantially all the P-wave vectors that compose a P-wave loop in three dimensions. A P-Elevation (P-El), shown also in FIG. 6b, is a parameter for the angle that the P-main vector forms in the vertical plane.

A P-change vector angle (PC-VA) denotes the angle difference between the main vector of the P-wave examined and the reference P-wave. A P-VM-parameter is the P-vector magnitude and a P-change vector magnitude (PC-VM) is the magnitude of the vector point of the reference P-main vector to the examined P-main vector. A P-QRS-vector angle (PQRS-VA) expresses the angle between the P and the QRS-main vectors. A P-QRS-change vector angle (PQRSC-VA) expresses the upcoming changes compared to the values of the reference P- and QRS-waves.

A PQ-vector magnitude (PQ3-VM) describes the PQ-magnitude in a three-dimensional environment. A PQ-change vector magnitude (PQ3 C-VM) is the distance of the vector point of the reference P-main vector to the examined P-main vector magnitude in a three-dimensional environment.

In addition, angles of the PQ-vector (PQ-Az, PQ-El) and the difference (PQC-VA) between the PQ-wave examined and the main vector of the reference PQ-wave are calculated.

A P-vector loop length (P-VLL) is the perimeter of the loop drawn by the P-wave vector in a three-dimensional space. A P-vector loop velocity (P-VLV) describes the speed of the development of the P-wave vector loop length.

Magnitude ECG:

The parameters of the magnitude ECG are a P-vector magnitude area and the difference of it. FIG. 7 illustrates the P-wave in magnitude ECG. The P-vector magnitude area (P-MA) is an area of the P-wave in the magnitude ECG-signal. A P-vector magnitude area difference (PC-MA) describes the change between the examined P-wave magnitude area and the reference P-wave magnitude area:
PC-MA=sqrt[(Aₑₓₐₘ−A_{ref})²]

In addition, the PQ-parameters are calculated similar to the one-dimensional environment. A PQ-MVM describes the PQ-magnitude and a PQ-MA describes the PQ-area of the PQ-elevation. PQC-MA describes correspondingly the upcoming changes of the PQ-area compared to the values of the reference P-wave.
PQC-MA=sqrt(Aₑₓₐₘ−A_{ref})²

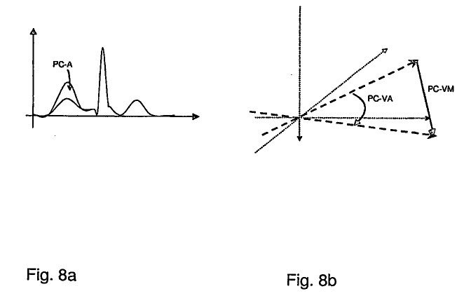

FIG. 8a represents the change of the P-wave area (PC-A) and FIG. 8b represents the change vector angle (PC-VA), as well as the change vector magnitude (PC-VM).

Parameters described above are preferably also used with the analysis of the beat stored into the atrium extra systole database.

When parameters have been calculated, the results are displayed (215). According to the invention, the results are displayed from every averaged time interval as a new point in trend curve over time. An advantage of this presentation is that the results are easier to see and a conclusion is easier to draw from the changes of the P-wave.

Analysis of the Parameters:

Parameters that concern the PQ-segment can have a great impact when studying the development of an acute myocardial infarction of atria. It is known that the manifestations of atrial infarction may include elevation or depression of the Ta-segment (Ta represents atrial repolarisation like the ST-segment and T-wave represent ventricular repolarisation). The Ta-segment is usually obscured by the QRS-complex and the early part of the ST-segment, but it's abnormalities may affect the PQ-segment. The parameters PQ-VM, PQ-A, PQC-A, PQ3-VM, PQ3 C-VM, PQ-MVM, PQ-MA, PQC-MA, PQ-Az, PQ-El and PQC-VA are developed to analyze these changes.

Parameters that concern the P-wave primary (Aₗ) and secondary (Aₗₗ) loop are developed most of all to evaluate the P-wave changes in heart insufficiency. It is known that in heart insufficiency the P-wave may become peaked or notched, depending on the etiology of disease. Although the specificity of these changes varies, it is a common opinion that in the left atrial enlargement due to heart insufficiency the P-wave became notched and has a negative terminal part, so that the P-wave has the appearance of the “fallen S”. The size of a negative terminal part may correlate to the level of the left atrial enlargement and heart insufficiency. The parameters P-AD and P-LAD are developed to analyze these dynamic changes.

Parameters that concern the QRS-complex vector angle and its relation to the P-wave vector angle are developed to discover if the change of P-wave vector angle is due to change of the heart position. The parameters PQRS-VA and PQRSC-VA are developed to analyze these changes.

Most of the parameters that concern the P-wave and PQ-segment area, the vector magnitude and angle, the PQ-time and the P-duration, the length and the velocity of the vector loop describe the character of P-wave vector loop widely. Their changes reflect the condition of the P-wave dynamically. For example, in the acute myocardial infarction of atria these parameters will change due to the tissue damage of the atrium.

There is some evidence that in the 12-lead ECG the P-wave duration and dispersion (difference between the longest and shortest P-wave duration) may be greater in persons who are in a higher risk to develop atrium fibrillation. Although the specificity of these changes varies, there is some evidence that when these changes decrease with the medication, the risk also decreases. As these changes in a 3-dimensional environment can be seen as increased irregularity of the P-wave vector loop, the above-mentioned parameters describe these dynamic changes ideally. Parameters are suitable also in other situations where dynamic changes in intra-atrial pressure, volume or conduction alter the P-wave configuration slowly. In addition, these parameters are useful in investigation of the quality atrial extra systoles.

System:

A cardiac analysis system according to the invention comprises means for signal processing and counting parameters. The system is adapted to take raw ECG-signal from existent datasystem and to store it into a file. Said cardiac analysis system is arranged to convert the sample file into a binary file containing X, Y, Z-samples. The samples in the generated file are preferably 16 bits signed integers. Samples are advantageously ordered in X(i), Y(i), Z(i), X(i+1), Y(i+1), Z(i+1), wherein X, Y, Z refers to the three orthogonal components of the VCG-signal and “i” refers to the beat number.

An input-file also comprises information about the resolution of the system, samples taken per second, data type (preferably signed 16 bit integer), beginning of the time frame to be analyzed related to the whole duration of the file, end of the time frame to be analyzed and number of channels (preferably three, corresponding to the orthogonal axes X, Y, Z). Naturally, the input-file can include other information as well.

The system also comprises means for preprocessing to remove the noise of the signal and to improve the SNR. Preprocessing is preferably done by filtering, which can be implemented with both low and high frequency of the bandpass preprocessing filter. The system also comprises a structure, which focuses on the beat specifications. Preferably, specification is done by measuring time and duration of the peaks and the waves. It can be adapted to measure the previous R-peak, the posterior R-peak, as well as the beginning of the range to calculate the baseline. Also, it can be stored with information of the channel used for R-peak detector and baseline correction, the rate to calculate the threshold for R-peak detection, and the duration of every beat in samples.

The detected beat is stored into the system. Information of the beat, such as a sample number when the “i”:th beat starts in the entire input file; a sample number of the ending of the “i”:th beat in the input file; duration of the beat in samples; wave form signal of the stored beat; baseline value of the “i”:th beat; duration of the beat in seconds; location of the R-wave peak in samples; validity of the P-wave (0 or 1) determined preferably by duration; P-wave onset; P-wave offset and length of the P-wave in samples are preferably also stored into the system.

A P-wave detector of the system is capable of validating the P-wave by knowing the threshold of covariance of the template method and the minimum and the maximum P-wave durations in samples. The validity of the P-wave is determined by measuring P-wave duration, which should settle between the minimum and the maximum values.

The system is preferably implemented as hardware and software. Therefore the system also comprises a computer program for implementing the method according to the invention. The computer program comprises computer readable code for acquiring the ECG data, detecting the P-wave from said data and analyzing said P-wave according to the method of the invention.

The method and the system describe the preferred embodiments of the P-wave analysis according to the invention. The main idea in the method is to analyze the dynamic changes of the P-wave in time. Implementation of the system can be carried out in different ways. For example it will be appreciated that in some cases other ECG-model is utilized, e.g. ECG-data can be acquired from a monitoring system using 10-lead arrangement, and determines an ECG-model that corresponds to the 12-lead arrangement to which the analysis is focused. Similarly, other parts of the ECG-model can be included to the analysis, e.g. the PQ-segment as described earlier.

By knowing this, it is obvious that the present invention is not limited to the description, but to claims discussed herein below.

1-33. (canceled)

34. A method for a cardiac analysis, the method comprising:
acquiring an ECG-signal;
detecting at least one wave of the ECG-signal; and
calculating parameter values of said wave, wherein said wave is a P-wave,
whereupon the cardiac analysis is focused to dynamic changes of the configuration of the P-wave, wherein substantially every detected P-wave is compared to a reference P-wave in defined time period.

35. The method according to claim 34, wherein the cardiac analysis is focused also to dynamic changes of the PQ-segment.

36. The method according to claim 34, wherein the ECG-signal is in the form of a vectorcardiogram.

37. The method according to claim 34, wherein a beat between two R-peaks is examined, whereupon said beat is classified into groups depending on whether the beat is having a duration between the predetermined time limit or the beat is having a duration under the predetermined time limit, whereupon both said beats are analyzed separately.

38. The method according to claim 34, wherein the P-wave is detected by a template method.

39. The method according to claim 34, wherein the P-wave is detected by a pattern recognition method.

40. The method according to claim 34, wherein the detected P-wave is stored in X, Y, Z leads.

41. The method according to claim 34, wherein the detected P-wave is averaged in the predetermined time interval.

42. The method according to claim 41, wherein at least one averaged P-wave is used as an initial reference P-wave, where the upcoming averaged P-waves are compared to.

43. The method according to claim 34, wherein at least one loop of the P-wave is detected.

44. The method according to claim 34, wherein the parameters of the P-wave in one-dimensional diagram are one or more of the following: the vector area, vector change area, P-area duplicity, PQ-vector magnitude, PQ-area and PQ change area.

45. The method according to claim 34, wherein the parameters of the P-wave in two-dimensional diagram are one or more of the following: the vector loop area, vector change loop area and P loop area duplicity.

46. The method according to claim 34, wherein the parameters of the P-wave in three-dimensional diagram are one or more of the following: the vector loop area, the vector change loop area, the angles of the azimun, the elevation, change vector, the P-QRS vector as well as the vector magnitude, change vector magnitude.

47. The method according to claim 34, wherein the parameters of the P-wave in magnitude environment are one or more of the following: the vector magnitude area, the vector change magnitude area difference and the vector magnitude.

48. The method according to claim 34, wherein the method comprises also the calculations of one or more of the following: the PQ-time, P-wave duration (P-dur), the length of the P-wave, the velocity of the P-wave vector loop.

49. The method according to claim 34, wherein the ECG-signal is acquired from a Frank system or a 12-lead ECG-arrangement.

50. The method according to claim 34, wherein the ECG-signal is acquired from a data storage unit that is independent of this invention and commercially available.

51. The method according to claim 34, wherein results of the parameters are displayed in a trend curve.

52. A cardiac analysis system, comprising:
first means for acquiring the ECG-signal;
second means for detecting at least one wave from the ECG-signal;
third means for calculating parameter values of said wave, wherein said wave is a P-wave, whereupon the cardiac analysis system is adapted to focus to dynamic changes of the configuration of the P-wave; and
means for comparing substantially every detected P-wave to a reference P-wave in defined time period.

53. The system according to claim 52, being further adapted to focus to dynamic changes of the PQ-segment.

54. The system according to claim 52, wherein the ECG-signal is in form of a vectorcardiogram.

55. The system according to claim 52, being also adapted to measure a duration of the beat between two R-peaks, wherein the system is also configured to compare the beat to the predetermined time limit and classified the beat into the one of two groups depending on whether the duration is between the predetermined time limit or under the predetermined time limit, wherein the system is also configured to analyze both groups separately.

56. The system according to claim 52, being adapted to detect the P-wave by a template method.

57. The system according to claim 52, being adapted to detect the P-wave by a pattern recognition method.

58. The system according to claim 52, being adapted to store the detected P-wave in X, Y, Z leads.

59. The system according to claim 52, being adapted to average the detected P-wave in the predetermined time interval.

60. The system according to claim 52, being adapted to use the first averaged P-wave as a reference P-wave and to compare the upcoming averaged P-waves to it.

61. The system according to claim 52, being adapted to detect at least one loop of the P-wave.

62. The system according to claim 52, being adapted to acquire the ECG-data from a Frank system or a 12-lead ECG-arrangement.

63. The system according to claim 52, being adapted to acquire the ECG-signal from a data storage unit that is independent of this invention and commercially available.

64. The system according to claim 52, being adapted to display results of the parameters calculated in trend curve.

65. A computer program product, comprising:
a computer readable storage medium on which is stored a computer program code for a cardiac analysis, which computer program code comprises first computer instructions configured to acquire the ECG-signal, second computer instructions configured to detect at least one wave from the ECG-signal and third computer instructions configured to calculate parameter values of said wave, wherein said wave is P-wave, whereupon the computer program code has instructions for focusing to the dynamic changes of the configuration of said P-wave, wherein said computer program code additionally comprises computer instructions configured to compare substantially every detected P-wave to a reference P-wave in defined time period.

66. The computer program product according to claim 65, wherein, the cardiac analysis is focused also to dynamic changes of the PQ-segment.
